(19) United States
(12) Patent Application Publication
(10) Pub. No.: US 2013/0345534 A1
(43) Pub. Date: Dec. 26, 2013

(54) Electrodes with Conductive Polymer Underlayer

(71) Applicant: Abbott Diabetes Care Inc., (US)

(72) Inventors: Udo Hoss, Castro Valley, CA (US); Adam Heller, Austin, TX (US)

(73) Assignee: ABBOTT DIABETES CARE INC., Alameda, CA (US)

(21) Appl. No.: 13/705,973

(22) Filed: Dec. 5, 2012

Related U.S. Application Data
(60) Provisional application No. 61/568,834, filed on Dec. 9, 2011.

(51) Int. Cl. A61B 5/1468

(52) U.S. Cl. CPC A61B 5/1468
USPC 600/345

(57) ABSTRACT

The disclosure provides method and materials for preparing biosensors (e.g., in vitro test strips and in vivo sensors) with improved mechanical properties. In some aspects, for example, the electrochemical sensors have improved durability and are better able to withstand mechanical and electrochemical stresses such as those encountered during manufacturing, transportation, storage, and use (e.g., in vivo positioning, in vivo operation, or in vitro operation). Also for example, in some aspects the electrochemical sensors are less susceptible to pinholes and other manufacturing defects that degrade performance in traditional sensors.

### CROSS-REFERENCE TO RELATED APPLICATIONS

This application claims priority based on U.S. Provisional Application No. 61/568,834, filed Dec. 9, 2011, the disclosure of which is incorporated by reference herein.

### BACKGROUND

In vivo monitoring of the level of analytes such as glucose, lactate, or oxygen, is an important aspect for maintaining health in certain individuals. For example, high or low levels of glucose or other analytes may have detrimental effects, particularly in diabetics. A variety of analyte monitoring devices have been developed for continuous or automatic monitoring of analytes, such as glucose, in a patient's blood stream or interstitial fluid. A number of these devices use electrochemical sensors which are directly positioned in a blood vessel or in the subcutaneous tissue of the patient.

Many of the analyte monitoring devices use electrochemical sensors having electrodes and electrical traces that are printed or otherwise deposited onto a substrate. The electrodes may be loaded with sensing chemistry (e.g. enzymes) or function as counter/reference electrodes. The electrical traces connect the electrodes to other hardware components such as a meter or other control unit.

The materials used for the electrodes and traces typically must provide good electron transport from the sensing chemistry to the sensors and, ultimately, to the control unit. Also, especially in the case of electrodes positioned in vivo, the electrodes and conductive traces should not break or otherwise fail when exposed to mechanical stress over days or weeks. Many current electrochemical sensors lack sufficient durability to withstand the stresses that are commonly encountered for such devices.

### SUMMARY

The disclosure provides method and materials for preparing biosensors (e.g., in vitro test strips and in vivo sensors) with improved mechanical and electrochemical properties. In some aspects, for example, the electrochemical sensors have improved durability and are better able to withstand mechanical stresses and corrosion causing reactions such as those encountered during manufacturing, transportation, storage, and use (e.g., in vivo positioning (e.g., full or partial implantation, such as in a subcutaneous layer, etc.), in vivo operation, or in vitro operation). Also for example, in some aspects the electrochemical sensors are less susceptible to performance-affecting pinholes and other manufacturing defects that degrade performance in traditional sensors.

In some aspects, the disclosure provides an electrode assembly comprising: a substrate; a layer of a conductive material comprising a metal, metal oxide, or carbon; and a layer of a conductive polymeric material disposed between the substrate and the layer of conductive material.

In some aspects, the disclosure provides an electrode assembly comprising: a substrate; and a working electrode, the working electrode comprising: a layer of a conductive material; and a layer of a conductive polymeric material, wherein the layer of conductive polymeric material is disposed between the substrate and the layer of conductive material.

In some aspects, the disclosure provides a biosensor for detecting an analyte, the biosensor comprising a multilayer electrochemical sensor, the multilayer electrochemical sensor comprising a substrate, a layer of conductive polymeric material disposed on the substrate, and a layer of conductive material disposed on the layer of conductive polymeric material.

In some aspects, the disclosure provides a method for manufacturing an electrode assembly, the method comprising forming an electrode pattern in a multilayer structure, the multilayer structure comprising an intrinsically conductive polymer (ICP) disposed on a substrate and a conductive material disposed on the ICP, wherein the conductive material is selected from metals, conductive metal oxides, and conductive forms of carbon.

### BRIEF DESCRIPTION OF THE FIGURES

A detailed description of various embodiments of the present disclosure is provided herein with reference to the accompanying drawings, which are briefly described below. The drawings are illustrative and are not necessarily drawn to scale. The drawings illustrate various embodiments of the present disclosure and may illustrate one or more embodiment(s) or example(s) of the present disclosure in whole or in part. A reference numeral, letter, and/or symbol that is used in one drawing to refer to a particular element may be used in another drawing to refer to a like element.

FIG. 1A is a plan view of an electrochemical sensor prepared according to an embodiment provided herein. The sensor has two electrodes disposed on a substrate.

FIG. 1B is a cross sectional view of the electrochemical sensor shown in FIG. 1a.

FIG. 2A is a plan view of an electrode and trace according to an embodiment provided herein. Two layers of conductive material having differing dimensions are shown.

FIG. 2B is a cross sectional view of an electrochemical sensor employing the electrode and trace shown in FIG. 2A.

FIG. 3A is a cross sectional view of an electrochemical sensor having three electrode traces. Each trace includes two layers of conducting material, and the two layers have differing dimensions such that the underlying layer is smaller in area than the overlaying layer.

FIG. 3B is a cross sectional view of an electrochemical sensor having three electrode traces. Each trace includes two layers of conducting material. The underlying layer is recessed into the substrate, and the two layers have the same dimensions.

FIG. 4 is a plan view of an electrochemical sensor according to an embodiment provided herein. The sensor has two electrodes, each made of two layers of conductive material. The underlying layer of conductive material covers a significantly larger area of the substrate compared with the overlaying layer of conductive material.

FIG. 5 is a plan view of an electrochemical sensor according to an embodiment provided herein. The sensor has three electrodes, three electrode traces, and three electrical contacts.

FIG. 6 is a plan view of an electrochemical sensor according to an embodiment provided herein. The sensor has three electrodes. Three electrode traces are also shown in the figure.

FIG. 7 is a graph showing current v. analyte concentration for a variety of sensors prepared according to embodiments provided herein.

### DETAILED DESCRIPTION

The disclosure provides method and materials for preparing biosensors (e.g., in vitro test strips and in vivo sensors) with improved mechanical properties. In some aspects, for example, the electrochemical sensors have improved durability and are better able to withstand mechanical stresses such as those encountered during manufacturing, transportation, storage, and use (in vivo positioning (e.g., full or partial implantation, such as in a subcutaneous layer, etc.), in vivo operation, or in vitro operation). Also for example, in some aspects the electrochemical sensors are less susceptible to performance affecting pinholes and other manufacturing defects that degrade performance in traditional sensors.

Before the embodiments of the present disclosure are described, it is to be understood that this invention is not limited to particular embodiments described, as such may, of course, vary. It is also to be understood that the terminology used herein is for the purpose of describing particular embodiments only, and is not intended to be limiting, since the scope of the embodiments of the invention will be embodied by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

In the description of the invention herein, it will be understood that a word appearing in the singular encompasses its plural counterpart, and a word appearing in the plural encompasses its singular counterpart, unless implicitly or explicitly understood or stated otherwise. Merely by way of example, reference to “an” or “the” “analyte” encompasses a single analyte, as well as a combination and/or mixture of two or more different analytes, reference to “a” or “the” “concentration value” encompasses a single concentration value, as well as two or more concentration values, and the like, unless implicitly or explicitly understood or stated otherwise. Further, it will be understood that for any given component described herein, any of the possible candidates or alternatives listed for that component, may generally be used individually or in combination with one another, unless implicitly or explicitly understood or stated otherwise. Additionally, it will be understood that any list of such candidates or alternatives is merely illustrative, not limiting, unless implicitly or explicitly understood or stated otherwise.

Various terms are described below to facilitate an understanding of the invention. It will be understood that a corresponding description of these various terms applies to corresponding linguistic or grammatical variations or forms of these various terms. It will also be understood that the invention is not limited to the terminology used herein, or the descriptions thereof, for the description of particular embodiments. Merely by way of example, the invention is not limited to particular analytes, bodily or tissue fluids, blood or capillary blood, or sensor constructs or usages, unless implicitly or explicitly understood or stated otherwise, as such may vary.

The publications discussed herein are provided solely for their disclosure prior to the filing date of the application. Nothing herein is to be construed as an admission that the embodiments of the invention are not entitled to antedate such publication by virtue of prior invention. Further, the dates of publication provided may be different from the actual publication dates which may need to be independently confirmed.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are described herein. It is understood that the present disclosure supersedes any disclosure of an incorporated publication to the extent there is a contradiction.

The term “typically” is used to indicate common practices of the invention. The term indicates that such disclosure is exemplary, although (unless otherwise indicated) not necessary, for the materials and methods of the invention. Thus, the term “typically” should be interpreted as “typically, although not necessarily.” Similarly, the term “optionally,” as in a material or component that is optionally present, indicates that the invention includes instances wherein the material or component is present, and also includes instances wherein the material or component is not present.

In further describing the present disclosure, electrodes and electrochemical sensors are described first in greater detail. Next, methods for manufacturing electrodes and electrochemical sensors are described. Subsequently, devices and systems practicing methods of the present disclosure are also described.

### Substrate

The electrochemical sensors of interest include a substrate. In some embodiments, the substrate includes a material selected from inert, non-conducting organic polymers, inorganic polymers, and combinations thereof. In some embodiments, the substrate is a material selected from plastics, ceramics, and combinations thereof. Examples of useful plastic or polymeric materials include thermoplastics such as polycarbonates, polyesters (e.g., Mylar® and polyethylene terephthalate (PET)), polyvinyl chloride (PVC), polyethylene (PE), ABS, polyethylene naphthalate (PEN), polystyrene, cellulose, triacetyl cellulose, polyurethanes, polyethers, polyamides, polyimides, copolymers including any of the abovementioned, such as PETG (glycol-modified polyethylene terephthalate), blends of any of the abovementioned, or the like. The substrate may include a coextruded film of any of the above mentioned materials. Examples of ceramics and inorganic polymers include oxides of metals such as aluminum, silicon, titanium, vanadium, and the like.

In some embodiments, the substrate is flexible. Suitable materials for a flexible substrate include the aforementioned polymer materials. In alternative embodiments, the substrate is rigid. Suitable materials for a rigid substrate include the aforementioned ceramic materials. The substrate may be prepared with any shape and thickness suitable for the desired application.

In some embodiments, an adhesion promoter may be disposed on the substrate to facilitate adhesion of the overlaying layers (traces, electrodes, etc. as described below) to the substrate. Examples of adhesion promoters include polyacrylates, polyolefins, epoxy compounds, polyesters, and the like. In some embodiments adhesion may be promoted by physical or chemical treatment of the substrate surface, such as exposure to a gaseous plasma of an inert gas, oxygen or other gas.

The thickness of the substrate can be selected as desired based on a number of factors such as material cost, flexibility and rigidity, intended use, and the like. For in vivo positionable electrochemical sensors, the substrate is thick enough to support the electrodes and traces but thin enough such that the electrochemical sensor is suitable for in vivo positioning. In some embodiments the substrate has a thickness greater than 50 μm, or greater than 100 μm, or greater than 300 μm, or greater than 500 μm. In some embodiments, the substrate has a thickness less than 500 μm, or less than 300 μm, or less than 100 μm, or less than 50 μm. In some embodiments, the substrate has a thickness between 50 and 500 μm, or between 100 and 300 μm.

In some embodiments, the substrate is not patterned and presents a smooth surface upon which other device components (e.g., electrodes, traces, etc.) are disposed. In other embodiments, the substrate is patterned with a network of channels; in such embodiments one or more components or component layers may be disposed partial or completely in the channels.

### Traces, Electrodes, and Contacts

The electrochemical sensors of interest include one or more electrodes. In some embodiments, the electrodes of the sensors of interest are disposed either partially or completely on the substrate. In some embodiments the sensors of interest include two electrodes, and in some embodiments the sensors of interest include more than two electrodes (such as three, four, five, or more electrodes). The sensors include electrodes selected from working electrodes, reference electrodes, counter electrodes, second electrodes, third electrodes, etc.

In some embodiments, the electrochemical sensors of interest further include traces (also referred to herein as “leads”) associated with the electrodes. For example, the working electrode is associated with a working electrode trace that provides an electrical connection between the working electrode and an electrical contact associated with the working electrode. Similarly, the counter electrode is associated with a counter electrode trace that provides an electrical connection between the counter electrode and an electrical contact associated with the counter electrode. In some embodiments, there are no electrical contacts associated with the electrodes, and the traces provide direct electrical connections between the electrodes and a control unit or other component of the analyte sensing device. In other embodiments, the electrical contacts are present and are located (in whole or in part) on the substrate along with the electrodes and traces.

In some embodiments, the electrodes and traces (and electrical contacts, when present) include a conductive material layer comprising a conductive material, wherein the conductive material is selected from metals, metal alloys, conductive metal oxides, other conductive materials, and combinations thereof. For example, the electrodes and traces may include a metal such as gold, silver, platinum, ruthenium, palladium, nickel, zinc, and the like. Also for example, the electrodes and traces may include a metal oxide such as indium tin oxide (ITO), ruthenium dioxide, tin oxide, zinc oxide, or titanium dioxide, which materials may be doped as necessary to obtain conductivity. Also for example, the electrodes and traces may include a conductive form of carbon (e.g., graphite, graphene, nanotubes, etc.), which materials may be doped as necessary to obtain conductivity. In some embodiments, the conductive material has a shear modulus that is greater than 15 GPa, or greater than 20 GPa, or greater than 25 GPa. For example, the shear modulus of gold is about 27 GPa.

In some embodiments, the electrodes and electrode traces (and electrical contacts, when present) further include a conductive polymeric material layer comprising a conductive polymeric material, wherein the conductive polymeric material is selected from one or more intrinsically conductive polymer (ICP). Examples of suitable ICPs can include polyacetylene, poly(p-phenylene vinylene) (PPV), polythiophene, poly(3-alkylthiophene), poly(3,4-ethylenedioxythiophene) (PEDOT), poly(p-phenylene sulfide) (PPS), polyaniline (PANI), polypyrrole, polycarbazole, polyindole, polyazepine, polynaphthalene, polyazulene, polypyrene, polyphenylene, polyfluorene, and combinations, derivatives, and copolymers thereof. In some embodiments, the conductive polymeric material includes sulfur atoms. An example of a commercially available polymer is CurrentFine® (marketed by Tejijn Dupont). Another example of a suitable ICP is the combination PEDOT:PSS, such as in dispersions thereof. Furthermore, an example of a suitable sulfur-containing conductive polymer film is provided in U.S. Pat. No. 7,527,853, the relevant portion of which is incorporated herein by reference. Any such materials as described herein may be doped as needed in order to provide the desired level of conductivity. In some embodiments, the conductive polymeric material has a shear modulus that is less than 10 GPa, or less than 5 GPa, or less than 3 GPa, or less than 1 GPa. For example, the shear modulus of PEDOT has been reported at around 2.5 GPa. In some embodiments, the conductive polymeric material is not a conductive ink. For example, the conductive polymeric material is not an ink suitable for printing (e.g., via an ink jet printer).

In some embodiments, the conductive material and conductive polymeric material are present in the form of layers disposed on the substrate. In some embodiments, the conductive polymeric material layer is interposed between the conductive material layer and the substrate. In other embodiments, the conductive material layer is interposed between the conductive polymeric material layer and the substrate. Together, the conductive material layer and conductive polymeric material layer form a bilayer (also referred to as a multilayer) structure. The multilayer structure may further include additional layers, including additional layers of the conductive material and/or conductive polymeric materials. For example, in some embodiments the multilayer structures include two or more types of conductive polymer, wherein each type of conductive polymer is deposited as a layer of conductive polymeric material and together the layers form a composite layer of conductive polymeric material. As described in more detail below, each of the layers in the multilayer structures is patterned, and the pattern used for the various layers may be the same or different. Throughout this disclosure, the term “multilayer structure” is used to refer to the collection of conductive, patterned layers that is disposed on a substrate to form the one or more electrodes, traces, etc. of an electrochemical sensor.

In some embodiments, the conductive material is chemically bonded to the conductive polymeric material. Such chemical bonds may be present strictly between (i.e., bridging) the conductive material layer and conductive polymeric material layer, and/or such chemical bonds may be present within the layers (e.g., where material from one layer extends into the adjacent layer). The chemical bonds may be selected from one or more of the following types of bonding: covalent, ionic, hydrogen, and van der Waals. For example, in some embodiments, covalent bonds are formed and are present between the material of the conductive material layer and the conductive polymeric material layer. As a specific example, in some embodiments, the conductive polymeric material includes sulfur atoms (e.g., polythiophenes), and the conductive material is gold. The gold layer forms chemical bonds to the sulfur atoms in the underlayer of conductive polymeric material. Such chemical bonds provide improved durability and film performance.

As mentioned previously, in some embodiments, the conductive material layer is disposed on top of the conductive polymeric material layer, and the conductive polymeric material layer is disposed on top of the substrate. The conductive polymeric material layer is an ICP that is deposited such that it has conductivity greater than 0.1 S/cm, or greater than 1 S/cm, or greater than 10 S/cm, or greater than 100 S/cm. In some embodiments, the conductive material layer is deposited over the layer of ICP material, and the presence of the conductive material layer does not significantly reduce the conductivity of the conductive polymeric material layer. For example, the presence of the conductive material layer does not reduce the conductivity of the conductive polymeric material layer by more than 5%, or by more than 10%, or by more than 15%, or by more than 20%, or by more than 25%. Thus, for example, an electrochemical sensor according to the disclosure can have a plurality of multilayer electrodes wherein the first (top) layer includes a metal (or other material identified above as suitable for the conductive material) and the second (bottom) layer includes an ICP with a conductivity of greater than 0.1 S/cm.

In some embodiments, the shear modulus of the conductive material is significantly greater than the shear modulus of the conductive polymeric material. For example, in some such embodiments the shear modulus of the conductive material is greater than 2 times, or greater than 3 times, or greater than 5 times, or greater than 10 times the shear modulus of the conductive polymeric material. Because of the much higher shear modulus, the conductive material is more rigid and prone to introduction of defects (such as cracks) under strain compared with the conductive polymeric material. In some embodiments, the difference in shear modulus between the conductive material and the conductive polymeric materials is at least 5 GPa, or at least 10 GPa, or at least 15 GPa, or at least 20 GPa.

In some embodiments, the Young's modulus (Modulus of Elasticity) of the conductive material is significantly greater than the Young's modulus of the conductive polymeric material. For example, in some such embodiments the Young's modulus of the conductive material is in the range of about 10 to 200 GPa, or in the range of about 25 to about 100 GPa. In some embodiments the Young's modulus of the conductive material is greater than 10, or greater than 20, or greater than 30, or greater than 40, or greater than 50, or greater than 60, or greater than 70, or greater than 80, or greater than 90, or greater than 100 GPa. In some embodiments, the Young's modulus of the conductive polymeric material is in the range of about 0.01 to 8 GPa, or in the range of about 0.05 to about 5 GPa. In some embodiments, the Young's modulus of the conductive polymeric material is less than about 8, or less than about 5, or less than about 3, or less than about 1, or less than about 0.5, or less than about 0.1, or less than about 0.05 GPa. In some embodiments, the Young's modulus of the conductive material is at least 2 times greater, or at least 3 times greater, or at least 5 times greater, or at least 10 times greater, or at least 25 times greater, or at least 50 times greater than the Young's modulus of the conductive polymeric material. For example, the Young's modulus of PEDOT/PSS is approximately between 1 GPa and 2.7 GPa. Also for example, the Young's modulus of gold is approximately 69 GPa. In an embodiment using gold as conductive material and PEDOT/PSS as conductive polymeric material, the Young's modulus ratio would be between 25 and 69 (gold:PEDOT/PSS)

As mentioned above, in some embodiments, an adhesion promoter or adhesive material is present in the layered structure forming the electrodes and traces. For example, an adhesive material is present and is disposed between the conductive polymeric material layer and the substrate. In such embodiments, the adhesion promoter is compatible with the conductive polymeric material. By “compatible” is meant that the presence of the adhesion promoter does not significantly alter the properties (e.g., the conductivity) of the conductive polymeric material or the conductive polymeric material layer. For example, where the conductive polymeric material layer is an ICP that requires a specific crystal orientation in order to be conductive, the presence of the adhesion promoter does not interfere with such orientation. Furthermore, in some embodiments the presence of the adhesion promoter does not alter the conductive properties of the conductive material or the conductive material layer.

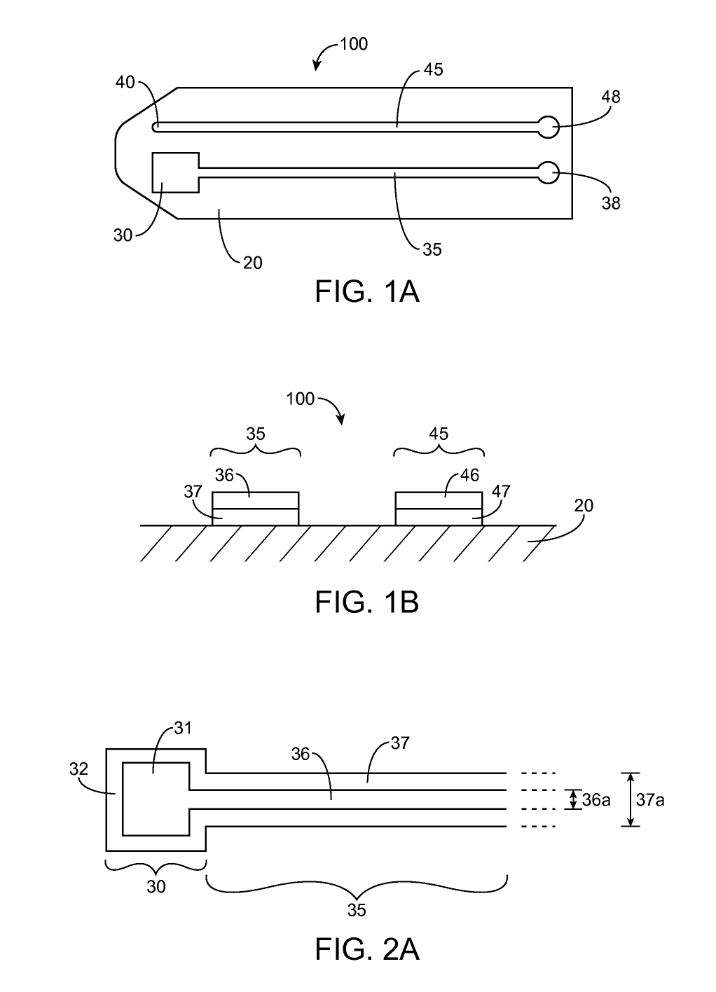

In some embodiments, the electrodes and traces form a pattern on the substrate, and the conductive material layer and conducting polymeric material layers are patterned identically to form such electrodes and traces. In such embodiments, the width and path of the traces, and the size and shape of the electrodes are patterned identically such that, in a top-down view of the electrochemical sensor, only the conductive material layer can be seen. An embodiment of this is illustrated in FIG. 1A. Electrochemical sensor 100 contains substrate 20, working electrode 30, working electrode trace 35, and electrical contact 38 associated with working electrode 30. Device 10 also contains counter electrode 40, counter electrode trace 45, and electrical contact 48 associated with counter electrode 40. In some such embodiments, at any given point and at every point of the traces on the substrate, the width of the conducting material layer and the width of the conducting polymeric material layer are exactly the same, or differ by less than 10%, or less than 5%, or less than 2%, or less than 1%. FIG. 1B shows a cross-sectional view of electrochemical sensor 100, which has traces 35 and 45 disposed on substrate 20. In trace 35, conductive material layer 36 has the same width as conductive polymeric material layer 37. In trace 45, conductive material layer 46 has the same width as conductive polymeric material layer 47. It will be appreciated that a similar cross section would be observed for electrodes having conductive material layer and conductive polymeric material layers that are patterned identically.

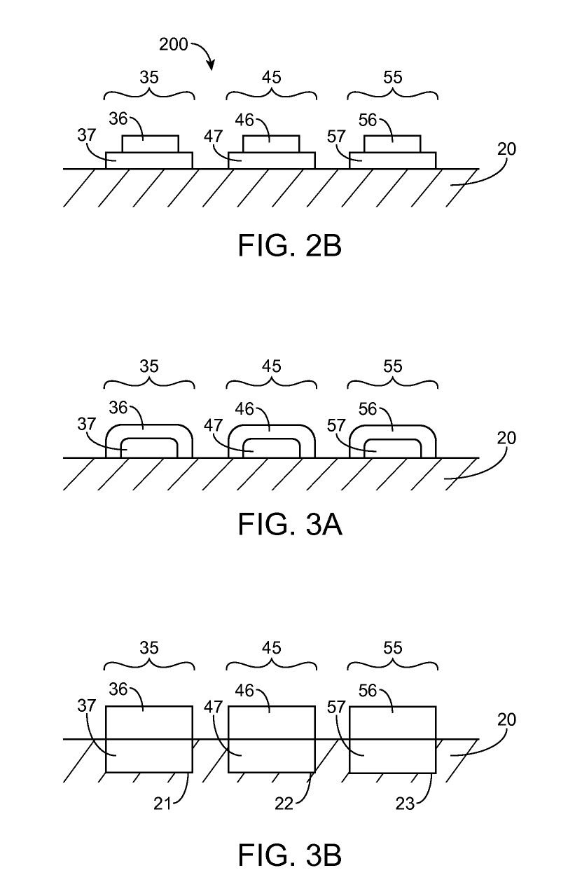

In some embodiments, the electrodes and traces form a pattern on the substrate, and the conducting material layer and conductive polymeric material layers are not patterned identically. Non-identical patterning as applied herein includes differences in pattern design, scale, and combinations thereof. In some such embodiments, for example, the lateral dimensions of the electrodes and traces are greater for the conductive polymeric material layer compared with the conductive material layer. This creates a “shadow” effect whereby, in a top-down view of the electrochemical sensor, the conductive polymeric material layer extends beyond and effectively outlines the conductive material layer. This is illustrated in FIG. 2A. Working electrode 30 is connected to working electrode trace 35. Working electrode 30 includes conductive material layer 31, and conductive polymeric material layer 32 disposed below (and having greater lateral dimensions than) conductive material layer 31. Working electrode trace 35 includes conductive material layer 36, and conductive polymeric material layer 37 disposed below (and having greater lateral dimensions than) conductive material layer 36. The width of the conductive material layer 36 and the width of the conductive polymeric material layer 37 are depicted by 36a and 37a, respectively. FIG. 2B shows a cross-sectional view of electrochemical sensor 200, which has traces 35, 45, and 55 disposed on substrate 20. As can be seen, in trace 35 conductive material layer 36 has a width narrower than conductive polymeric material layer 37. In trace 45, conductive material layer 46 has a width narrower than conductive polymeric material layer 47. In trace 55, conductive material layer 56 has a width narrower than conductive polymeric material layer 57. In some such embodiments, at any given point along the traces on the substrate (such as the cross section shown in FIG. 2B), the width of the conductive polymeric material layer is at least 10% greater, or at least 25% greater, or at least 50% greater, or at least 75% greater, or at least 100% greater than the width of the conductive material layer. For example, in some embodiments the conductive material layer portion of the traces (e.g., working electrode trace and counter electrode trace) for an electrochemical sensor have an overall average width of w1 on the substrate. The conductive polymeric material layer portion of the same traces has an overall average width that is greater than w1, such as up to 1.1*w1, or up to 1.25*w1, or up to 1.5*w1, or up to 1.75*w1, or up to 2*w1, or greater than 2*w1.

In some embodiments of non-identical patterns in the conductive material and conducting polymer material layers, the in-plane dimensions of the electrodes and traces are greater for the conductive material layer compared with the conductive polymeric material layer. This creates a “blanket” effect whereby the overlayer of conductive material partially or wholly encapsulates the underlayer of conductive polymeric material. A cross section of such a device is illustrated in FIG. 3A. Electrode trace 35 includes conductive material layer 36 and conductive polymeric material layer 37, and is shown disposed on (and entirely above the surface of) substrate 20. Similarly, electrode trace 45 includes conductive material layer 46 and conductive polymeric material layer 47, and electrode trace 55 includes conductive material layer 56 and conductive polymeric material layer 57.

In FIG. 3B, electrode trace 35 also includes conductive material layer 36 and conductive polymeric material layer 37, but conductive polymeric material layer 37 is provided within channel 21 formed in substrate 20. Similarly, electrode trace 45 includes conductive material layer 46 and conductive polymeric material layer 47, and electrode trace 55 includes conductive material layer 56 and conductive polymeric material layer 57, wherein conductive polymeric material layers 47 and 57 are disposed within channels 22 and 23, respectively. In FIG. 3B, conductive material layers 36, 46, and 56 are shown as having identical widths compared with conductive polymeric material layers 37, 47, and 57, respectively. It will be appreciated, however, that conductive material layers 36, 46, and 56 can alternatively have widths that are greater than or less than the widths of conductive polymeric material layers 37, 47, and 57

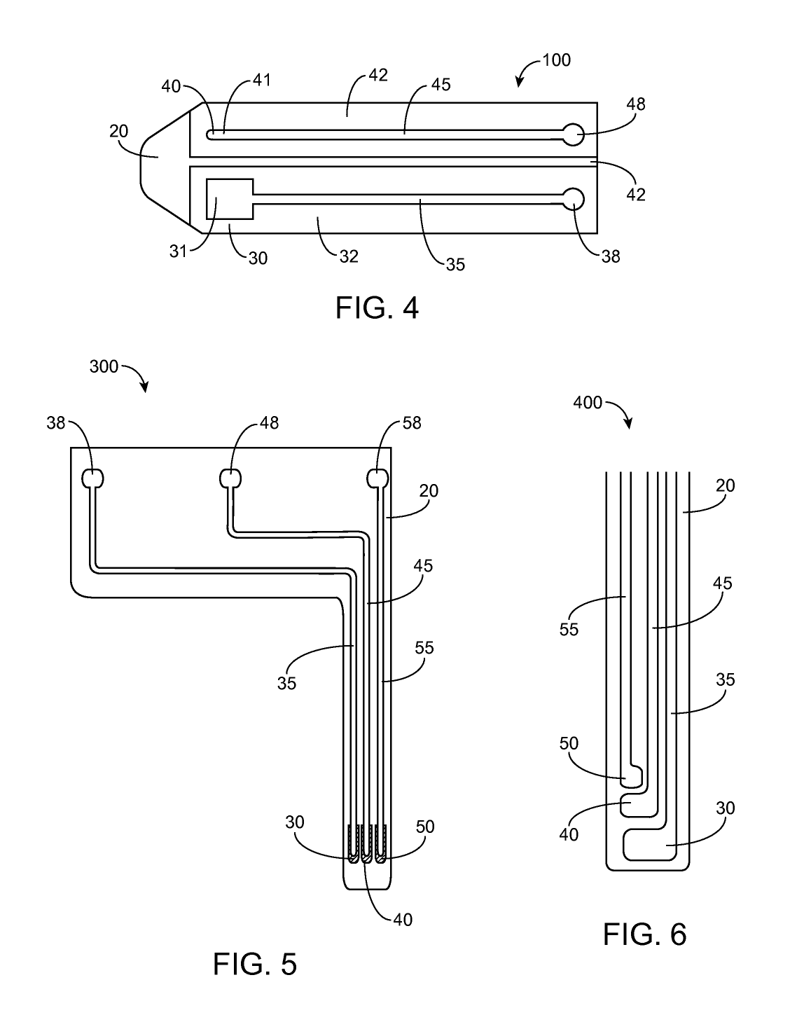

In still other embodiments of non-identical patterns in the conductive material layer and conductive polymeric material layers, the conductive polymeric material layer covers significantly greater area of the substrate, and contains relatively small breaks that are sufficient only to electrically isolate the electrodes. An example of this is illustrated in FIG. 4. Device 100 includes substrate 20. Conductive polymeric material layer 32 and conductive polymeric material layer 42 are disposed on substrate 20. Conductive material layer 31 is disposed on conductive polymeric material layer 32, and together these layers form working electrode 30. Similarly, conductive material layer 41 is disposed on conductive polymeric material layer 42, and together these layers form counter electrode 40. Conductive polymeric material layer 32 covers a significantly greater area of substrate 20 compared with conductive material layer 31. Similarly, conductive polymeric material layer 42 covers a significantly greater area of substrate 20 compared with conductive material layer 41. Gap 43 is present between (and electrically isolates) conductive polymeric material layer 32 and conductive polymeric material layer 42.

In some embodiments of non-identical patterns in the conductive material layer and conductive polymeric material layers, the two dimensional area of the conductive polymeric material layer is greater than the two dimensional area of the conductive material layer over the whole of the electrochemical sensor. In other embodiments, the two dimensional area of the conductive material layer is greater than the two dimensional area of the conductive polymeric material layer. For example, the area of one layer (i.e., either the conductive material layer or conductive polymeric material layer) can be up to 10% greater, or up to 25% greater, or up to 50% greater, or up to 75% greater or up to 100% greater, or more than 100% greater than the area of the other layer.

With reference to FIG. 5, device 300 is provided as an example electrochemical sensor configuration having three electrodes, traces, and electrical contacts. Thus, electrodes 30, 40, and 50 (which, as described herein, may be selected from working electrodes, reference electrode, counter electrode, third electrode, etc.) are in electrical communication with traces 35, 45, and 55, respectively, which in turn are in electrical communication with electrical contacts 38, 48, and 58, respectively.

With reference to FIG. 6, device 400 is provided as an example electrochemical sensor configuration having three electrodes and traces. Thus, electrodes 30, 40, and 50 (which, as described herein, may be selected from working electrodes, reference electrode, counter electrode, third electrode, etc.) are in electrical communication with traces 35, 45, and 55, respectively.

The thickness of the multilayer structures (i.e., the electrodes, traces, etc.) on the substrate is sufficient to provide the properties desired of the structures. In some embodiments, the multilayer structures have a thickness that is comparable to that of known electrodes, traces, etc. for in vivo positionable electrochemical sensors. In some embodiments, for example, the multilayer structures have thicknesses that are greater than 10 μm, or greater than 25 μm, or greater than 50 μm, or greater than 75 μm, or greater than 100 μm. In some embodiments, the multilayer structures have thicknesses that are less than 150 μm, or less than 100 μm, or less than 75 μm, or less than 50 μm, or less than 25 μm. In some embodiments, the multilayer structures have thicknesses that are in the range of about 10 μm to 200 μm, or in the range of about 12.5 to 75 μm, or in the range of about 25 to 50 μm. In some embodiments, the electrodes, traces, etc. of the devices of interest are slightly thicker than in monolithic (i.e., monolayer) devices. For example, whereas monolithic electrodes and traces may be in the range of about 10 μm to 100 μm, in some embodiments, the multilayer structures of interest have thicknesses that are in the range of about 20 to 200 μm, or about 30 to 150 μm.

In some embodiments, the thickness of the conductive material layer and the thickness of the conductive polymeric material layer are independently selected to provide sufficient conductivity and performance while minimizing material cost and electrode thickness. In some embodiments, the thickness of the conductive material layer is between about 1 μm and about 100 μm, or between about 5 μm and 75 μm, or between about 10 μm and 50 μm. In some embodiments, the thickness of the conductive material layer is less than 100 μm, or less than 75 μm, or less than 50 μm, or less than 40 μm, or less than 25 μm, or less than 20 μm, or less than 15 μm.

In some embodiments, the thickness of the conductive polymeric material layer is between about 1 μm and about 150 μm, or between about 10 μm and 100 μm, or between about 20 μm and 75 μm. In some embodiments, the thickness of the conductive polymeric material layer is less than 150 μm, or less than 125 μm, or less than 100 μm, or less than 750 μm, or less than 50 μm, or less than 40 μm, or less than 30 μm, or less than 25 μm. In some embodiments, when an adhesion promoter is present, such adhesion promoter adds a minimal or negligible amount to the thickness of the multilayer structure.

As mentioned in more detail below, devices having monolayer metal electrodes require electrode thicknesses that are sufficient to avoid pinholes. In some embodiments of the devices described herein, the thickness of the conductive material layer is less than the thickness of electrode layers commonly found such monolayer devices. For example, in some embodiments the conductive material layer includes a metal and has a thickness that is 20% less, or 40% less, or 50% less, or 60% less, or 75% less than the thickness of a similar electrode using a monolayer of metal (i.e., in the absence of an ICP underlay).

In some embodiments, it is desirable to ensure that materials such as dopants and the like do not leach out of the electrodes of interest, particularly while the electrodes are in use (i.e. in vivo). In some embodiments, the presence of the overlayer of conductive material prevents any such leaching. Also in some embodiments, the electrodes of interest are manufactured such that materials present in the electrodes are sufficiently immobilized such that they do not pose a biological threat for the intended use.

### Additional Components

In some embodiments, the devices of interest further include at least one electron transfer agent and/or at least one catalyst to facilitate the electrooxidation or electroreduction of the analyte. The electron transfer agent and/or catalyst may be incorporated into a sensing layer disposed on or near a working surface of the working electrode.

Suitable electron transfer agents, catalysts, and materials for sensing layers (and methods for incorporating such compounds and sensing layers into electrochemical sensors) are known and describe, for example, in U.S. Pat. Nos. 6,175,752; 5,665,222; 5,264,104; 5,356,786; 5,262,035; and 5,320,725, the relevant disclosures of which are incorporated herein by reference.

### Devices

In some embodiments, then, the disclosure provides analyte monitoring devices having one or more electrochemical sensors. In some embodiments, the analyte monitoring devices have additional hardware components such as one or more of the following: control units, display units, alarm units, interconnecting leads, and the like, or combinations thereof integrated into a single component. Additional information about such additional hardware components is described in U.S. Pat. No. 6,175,752, issued Jan. 16, 2001, the relevant portions of which disclosure are incorporated herein by reference.

As described in more detail throughout this disclosure, in some embodiments the devices of interest include electrochemical sensors (also referred to herein as “electrode assemblies”) that include a substrate, a layer of conductive material, and a layer of conductive polymeric material disposed between the substrate and the layer of conductive material. Also as described in detail herein, the layers of conductive material can be patterned (either identically or non-identically) and can form various components such as electrodes, traces, and electrical contacts.

In some embodiments, the electrochemical sensors of interest have increased durability and an improved ability to withstand mechanical stress compared with currently known electrochemical sensors. When an electrode of an electrochemical sensor is transiently or routinely poised at a potential where its metallic component could either corrode, or be hydrogen-embrittled, it is advantageous to replace it by a conductive polymeric material. Such is the case, for example, in a gold electrode on a polymer film where an intermediate layer of a second metal comprising, such as chromium, or cobalt, or nickel, or palladium comprising layer is applied to better adhere the gold to the polymer. The metals, are subject to corrosion and or hydrogen embrittlement, and their replacement by a non-corroding and/or not hydrogen embrittled conductive polymer material can be advantageous. Corrosion of the metallic intermediate layer undercuts the gold layer, causing its separation; hydrogen embrittlement of the intermediate metal and can cause cracking, the cracks propagating to the gold layer and even to the polymer. In either case, insulated or poorly connected electrode domains may form.

In some embodiments, the electrochemical sensors of interest include one or more multilayer electrodes and one or more multilayer traces connecting the electrodes with electrical contacts. In some embodiments, the electrochemical sensors of interest include one or more electrodes and traces that are each prepared from a plurality of conductive materials.

For example, in some embodiments the conductive polymeric material provides for bridging of electrical discontinuities in the conductive material layer. Such electrical discontinuities include manufacturing defects (e.g. pinholes, cracks, etc. caused during manufacturing, packaging, etc.) and cracks due to mechanical stress (e.g. cracks due to in vivo positioning or use in vitro or in vivo).

Throughout this description, devices having two electrodes (a working electrode and a counter electrode) are described, wherein the two electrodes have the same layered construction. It will be appreciated, however, that such devices are described merely as examples and for ease of illustration, and are not intended to limit the invention. For example, it will be appreciated that devices having more than two electrodes (e.g., a working electrode and separate reference electrode and counter electrode) are also suitable configurations. In addition, it will be appreciated that yet further configuration are also contemplates, such as, for example, sensors having one or more electrodes disposed on a first face of a substrate and one or more electrodes disposed on a second face of a substrate. Also for example, it will be appreciated that devices having electrodes with different configurations (e.g., a working electrode made of certain materials described herein and a counter electrode made of other materials described herein) are also suitable configurations.

### Methods of Manufacture

The electrochemical sensors of interest may be prepared using standard manufacturing techniques known in the art. The electrochemical sensors involve deposition of materials onto a substrate, and the substrate may be provided and prepared in advance of the deposition. In some embodiments, preparation of the substrate involves one or more of the following: cleaning of foreign matter, removal of a layer of substrate material to expose a fresh surface etching a pattern into the substrate, deposition of an adhesion promoter, and the like.

In some embodiments, the conducting polymeric material is deposited on the substrate in a manner suitable for deposition of an ICP. It will be appreciated that the phrase “on the substrate” includes instances where the ICP is deposited directly on the surface of the substrate as well as instances where the ICP is deposited indirectly on the surface of the substrate, such as when an adhesion promoter is disposed between the substrate and the conductive polymeric material layer. Examples of methods suitable for deposition of the ICP include vapor deposition, solution casting (e.g., spin coating, etc.), chemical vapor deposition, spray deposition, dip coating, and the like.

In some embodiments a curing or heating stage may be used to cause the ICP to form a conductive material or to increase conductivity of the layer. The curing stage may be carried out prior to any further manufacturing steps (i.e., immediately after deposition of the conductive polymeric material layer), or may be carried out after additional manufacturing steps have been completed (e.g., after deposition of the conductive material layer). In addition or in the alternative, in some embodiments, a doping step may be employed in order to obtain the desired level of conductivity in the ICP. As with the curing stage, the doping stage may be carried out either immediately after deposition of the ICP, or, where appropriate, after other manufacturing steps as described herein.

In some embodiments the ICP is deposited over the whole of the substrate, whereas in other embodiments a mask or other means for patterning the deposition is used in order to form a patterned conductive polymeric material layer.

In some embodiments, the conducting material is deposited on the conductive polymeric material layer in a manner suitable for deposition of a layer of the conducting material. For example, when the conducting material is gold or another metal, the conductive polymeric material layer may be deposited using a method such as sputtering, thermal evaporation, or electrochemical methods. The method may be selected based on a variety of factors. One such factor is the identity of the conductive polymeric material, and the method of deposition for the conductive material is selected so as not to significantly damage the conductive polymeric material layer. In some embodiments the conductive material is deposited over the whole area of the substrate, particularly when the ICP has also been deposited over the whole area of the substrate. In other embodiments, a mask or other means for patterning the deposition is used in order to form a patterned conductive material layer. Where patterning by masking is carried out in both the conductive material layer and conductive polymeric material layers, in some embodiments the same mask is used for both depositions. In other embodiments, a different mask is used for the two layers.

In some embodiments, after deposition of the conductive material layer and conductive polymeric material layer, a patterning step is carried out in order to form the electrodes, traces, etc. Where the conductive material layer and/or conductive polymeric material layers are deposited as patterned layers, a further patterning step may be carried out if it is desired to refine the shape of the electrodes and traces as formed. The patterning of the conductive material layer and conductive polymeric material layers can be carried out simultaneously after deposition of uniform layers covering the substrate in whole or in part.

Methods for patterning include ablation methods known in the art, such as via laser ablation, reactive ion etching (RIE), or scribing. The patterning of the multilayer structure can also be carried out in multiple steps. For example, in some embodiments, the conductive polymeric material layer is patterned immediately after deposition in a first patterning step, the conductive material layer is subsequently deposited, and the conductive material layer is then patterned in a second patterning step.

As mentioned herein, an adhesion promoter may be incorporated into the multilayer structure. In addition or in the alternative, sand blasting or another method for forming surface roughness can be used to improve adhesion of one layer disposed on another (e.g., of the conductive material layer on the conductive polymeric material layer, or of the conductive polymeric material layer on the substrate).

### Properties and Uses

In some embodiments, the electrochemical sensors described herein are suitable for use in an analyte measuring device. For example, the sensors are useful in a bioanalytical device for concentration measurements of biologically important analytes such as glucose, oxygen, and the like.

In some embodiments, the sensors of interest are useful as part of a system for in vivo measurement and monitoring of analyte concentrations. For example, the electrochemical sensors are suitable for in vivo positioning (in whole or in part) into an animal such as a human. In such embodiments, the sensors or systems to which they belong may include additional components to aid in vivo positioning, removal, and/or data collection. In some embodiments, in vivo positionable sensors according to the disclosure are suitable for continuous monitoring of analyte levels. In some embodiments, in vivo positionable sensors according to the disclosure are suitable for periodic and/or long term measuring of analyte levels.

In some embodiments, the sensors of interest are useful for in vitro measurement and monitoring of analyte concentrations. For example, the electrochemical sensors are suitable for use as analyte test strips. In some embodiments such test strips are part of a system for measuring analyte concentrations, such system including a device for reading data from the test strips. In some embodiments such test strips are designed for single use (i.e., disposable) and in other embodiments such test strips are designed for multiple use applications.

In some embodiments, the electrochemical sensors of interest have one or more improved properties over previously known sensors. For example, in some embodiments, the sensors of interest are less sensitive to defects such as pinholes in the electrode layer. Also for example, in some embodiments, the sensors of interest are less sensitive to fractures or other defects in the electrode layer (i.e., breaks in the continuity of the electrode layer typically caused by mechanical stress, manufacturing defects, or the manufacturing process itself). Without wishing to be bound by theory, it is believed that such improved performance of the electrochemical sensors described herein is due to the underlaying layer of ICP that provides a conductive pathway even when a defect or discontinuity such as a crack appears in the overlaying metal layer.

In previously known sensors employing metal electrodes, the sensors suffer from a tradeoff in terms of electrode thickness. Electrodes having thicker metal films are less flexible and tend to crack or peel with use or upon manufacturing. Such cracking and peeling decreases the utility of the device, particularly if it introduces electrical discontinuities. However, electrodes having thinner metal films are more likely to suffer from defects such as pinholes, thereby suffering decreased device performance. The devices disclosed herein employ multilayer electrodes and traces in order to reduce the deleterious effects of this tradeoff. The ICP underlayer and conducting material overlayer provide electrochemical sensors that maintain functionality even when pinholes, cracks, or other defects are present in the conducting material overlayer. In some embodiments, the devices disclosed herein can be subjected to the shear stresses of manufacturing (e.g., cutting, bending, etc.) as well as the shear stresses of in vivo use and yet still maintain sufficient conductivity in order to provide the desired analytical data. In some embodiments, the devices according to the disclosure are also less susceptible to defects caused by mismatched thermal coefficients of expansion between the substrate and the conductive material.

### Analyte Sensors

The present methods can be used to make a variety analyte in vitro test strips of any kind, size, or shape known to those skilled in the art; for example, FREESTYLE® and FREESTYLE LITE™ test strips, as well as PRECISION™ test strips sold by ABBOTT DIABETES CARE Inc. In addition to the embodiments specifically disclosed herein, the present methods can be employed with a wide variety of analyte test strips, e.g., those disclosed in U.S. patent application Ser. No. 11/461,725, filed Aug. 1, 2006; U.S. Patent Application Publication No. 2007/0095661; U.S. Patent Application Publication No. 2006/0091006; U.S. Patent Application Publication No. 2006/0025662; U.S. Patent Application Publication No. 2008/0267823; U.S. Patent Application Publication No. 2007/0108048; U.S. Patent Application Publication No. 2008/0102441; U.S. Patent Application Publication No. 2008/0066305; U.S. Patent Application Publication No. 2007/0199818; U.S. Patent Application Publication No. 2008/0148873; U.S. Patent Application Publication No. 2007/0068807; U.S. patent application Ser. No. 12/102,374, filed Apr. 14, 2008, and U.S. Patent Application Publication No. 2009/0095625; U.S. Pat. No. 6,616,819; U.S. Pat. No. 6,143,164; U.S. Pat. No. 6,592,745; U.S. Pat. No. 6,071,391 and U.S. Pat. No. 6,893,545; the disclosures of each of which are incorporated by reference herein in their entirety.

The present methods can be used to make a variety of in vivo analyte sensors of any kind, size, or shape known to those skilled in the art. In addition to the embodiments specifically disclosed herein, the present methods can be employed with a wide variety of in vivo analyte sensors, e.g., those disclosed in U.S. Pat. No. 6,175,752; U.S. Pat. No. 6,134,461; U.S. Pat. No. 6,579,690; U.S. Pat. No. 6,605,200; U.S. Pat. No. 6,605,201; U.S. Pat. No. 6,654,625; U.S. Pat. No. 6,746,582; U.S. Pat. No. 6,932,894; U.S. Pat. No. 7,090,756; U.S. Pat. No. 5,356,786; U.S. Pat. No. 6,560,471; U.S. Pat. No. 5,262,035; U.S. Pat. No. 6,881,551; U.S. Pat. No. 6,121,009; U.S. Pat. No. 7,167,818; U.S. Pat. No. 6,270,455; U.S. Pat. No. 6,161,095; U.S. Pat. No. 5,918,603; U.S. Pat. No. 6,144,837; U.S. Pat. No. 5,601,435; U.S. Pat. No. 5,822,715; U.S. Pat. No. 5,899,855; U.S. Pat. No. 6,071,391; U.S. Pat. No. 6,377,894; U.S. Pat. No. 6,600,997; U.S. Pat. No. 6,514,460; U.S. Pat. No. 5,628,890; U.S. Pat. No. 5,820,551; U.S. Pat. No. 6,736,957; U.S. Pat. No. 4,545,382; U.S. Pat. No. 4,711,245; U.S. Pat. No. 5,509,410; U.S. Pat. No. 6,540,891; U.S. Pat. No. 6,730,200; U.S. Pat. No. 6,764,581; U.S. Pat. No. 6,503,381; U.S. Pat. No. 6,676,816; U.S. Pat. No. 6,893,545; U.S. Pat. No. 6,514,718; U.S. Pat. No. 5,262,305; U.S. Pat. No. 5,593,852; U.S. Pat. No. 6,746,582; U.S. Pat. No. 6,284,478; U.S. Pat. No. 7,299,082; U.S. Pat. No. 7,811,231; U.S. Pat. No. 7,822,557; U.S. Pat. No. 8,106,780; U.S. Patent Application Publication No. 2010/0198034; U.S. Patent Application Publication No. 2010/0324392; U.S. Patent Application Publication No. 2010/0326842 U.S. Patent Application Publication No. 2007/0095661; U.S. Patent Application Publication No. 2008/0179187; U.S. Patent Application Publication No. 2008/0177164; U.S. Patent Application Publication No. 2011/0120865; U.S. Patent Application Publication No. 2011/0124994; U.S. Patent Application Publication No. 2011/0124993; U.S. Patent Application Publication No. 2010/0213057; U.S. Patent Application Publication No. 2011/0213225; U.S. Patent Application Publication No. 2011/0126188; U.S. Patent Application Publication No. 2011/0256024; U.S. Patent Application Publication No. 2011/0257495; U.S. Patent Application Publication No. 2012/0157801; U.S. patent application Ser. No. 13/407,617; U.S. patent application Ser. No. 13/526,136; U.S. Patent Application Publication No. 2012/0157801; and U.S. Patent Application Publication No. 2010/0213057; the disclosures of each of which are incorporated herein by reference in their entirety. Moreover, methods of the present disclosure may be practiced using battery-powered or self-powered analyte sensors, such as those disclosed in U.S. Patent Application Publication No. 2010/0213057.

Each of the various references, presentations, publications, provisional and/or non-provisional U.S. patent applications, U.S. patents, non-U.S. patent applications, and/or non-U.S. patents that have been identified herein, is incorporated herein by reference in its entirety.

Other embodiments and modifications within the scope of the present disclosure will be apparent to those skilled in the relevant art. Various modifications, processes, as well as numerous structures to which the embodiments of the invention may be applicable will be readily apparent to those of skill in the art to which the invention is directed upon review of the specification. Various aspects and features of the invention may have been explained or described in relation to understandings, beliefs, theories, underlying assumptions, and/or working or prophetic examples, although it will be understood that the invention is not bound to any particular understanding, belief, theory, underlying assumption, and/or working or prophetic example. Although various aspects and features of the invention may have been described largely with respect to applications, or more specifically, medical applications, involving diabetic humans, it will be understood that such aspects and features also relate to any of a variety of applications involving non-diabetic humans and any and all other animals. Further, although various aspects and features of the invention may have been described largely with respect to applications involving in vivo positioned sensors, such as transcutaneous or subcutaneous sensors, it will be understood that such aspects and features also relate to any of a variety of sensors that are suitable for use in connection with the body of an animal or a human, such as those suitable for use as in vivo positioned in the body of an animal or a human. Finally, although the various aspects and features of the invention have been described with respect to various embodiments and specific examples herein, all of which may be made or carried out conventionally, it will be understood that the invention is entitled to protection within the full scope of the appended claims.

The following examples are put forth so as to provide those of ordinary skill in the art with a complete disclosure and description of how to make and use the embodiments of the invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example

### In Vivo Sensor Having an Electrode with Conductive Polymer Underlayer

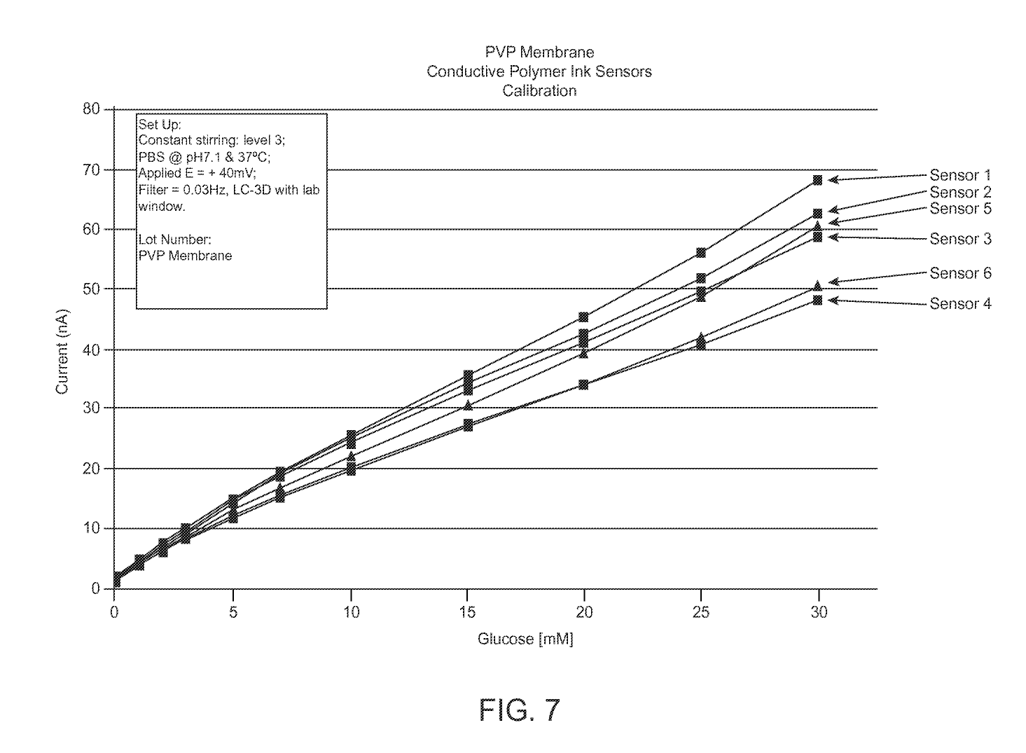

To test conductivity and ability of the layered electrode structure to provide adequate electron transfer capability, an in vivo sensor was constructed by depositing sensing chemistry (e.g., analyte responsive enzyme and polymerically bound redox mediator) on the conductive side (500 Ohm/square) of a strip of CurrentFine® (Teijin DuPont) film. The substrate was PET film having a thickness of 125 μm. Subsequently the film was dipped in a membrane polymer solution (e.g., poly-vinyl pyridine). The resulting strips (n=6) were then tested in glucose solutions up to 30 mM using separate reference (Ag/AgCl) and counter (carbon) electrodes. The resulting data are shown in FIG. 7. Based on the data in FIG. 7, the constructed sensors show good linearity over the whole range of glucose.

What is claimed is:

1. An electrode assembly comprising:
a substrate;
a layer of a conductive material comprising a metal, metal oxide, or carbon; and
a layer of a conductive polymeric material disposed between the substrate and the layer of conductive material.

2. The electrode assembly of claim 1, wherein the layer of conductive material and the layer of conductive polymeric material are patterned.

3. The electrode assembly of claim 2, wherein the pattern of the conductive material and the pattern of the conductive polymeric material are identical such that the two layers cover the same regions of the substrate and have the same two-dimensional area.

4. The electrode assembly of claim 2, wherein the pattern of the conductive material and the pattern of the conductive polymeric material are not identical such that the two-dimensional area of the layer of conductive material is greater than or less than the two-dimensional area of the layer of conductive polymeric material.

5. The electrode assembly of claim 1, wherein the conductive material has a Young's modulus that is greater than the Young's modulus of the conductive polymeric material.

6. The electrode assembly of claim 5, wherein the Young's modulus of the conductive material is at least 10 times greater than the Young's modulus of the conductive polymeric material

7. The electrode assembly of claim 1, wherein the layer of conductive polymeric material is disposed within channels patterned into the substrate.

8. The electrode assembly of claim 1, wherein the substrate is unpatterned and wherein the layer of conductive polymeric material is disposed on the substrate.

9. The electrode assembly of claim 1, wherein the conductive polymeric material provides for bridging of electrical discontinuities in the conductive material layer.

10. The electrode assembly of claim 9, wherein the electrical discontinuities include manufacturing defects and cracks due to mechanical stress.

11. The electrode assembly of claim 2, wherein the pattern comprises a working electrode, working electrode trace, counter electrode and a counter electrode trace, and optionally comprises one or more elements selected from a third electrode, a third electrode trace a working electrode electrical contact, a counter electrode electrical contact, and a third electrode electrical contact.

12. The electrode assembly of claim 1, wherein the conductive polymeric material is arranged such that no portion of the conductive material contacts the substrate.

13. The electrode assembly of claim 1, wherein a portion of the conductive material contacts the substrate.

14. The electrode assembly of claim 1, wherein the conductive material is selected from gold, silver, platinum, ruthenium, palladium, nickel, zinc, indium tin oxide (ITO), ruthenium dioxide, tin oxide, zinc oxide, or titanium dioxide, graphite, graphene, carbon nanotubes, and derivatives thereof, and wherein the conductive polymeric material is a doped or undoped intrinsically conductive polymer (ICP) having a conductivity greater than about 0.1 S/cm.

15. The electrode assembly of claim 11, wherein the average width of the layer of conductive material for the working electrode trace is w1, and wherein the average width of the layer of conductive polymeric material for the working electrode trace is greater than or equal to w1.

16. An electrode assembly comprising:
a substrate; and
a working electrode, the working electrode comprising:
a layer of a conductive material; and
a layer of a conductive polymeric material,
wherein the layer of conductive polymeric material is disposed between the substrate and the layer of conductive material.

17. The electrode assembly of claim 16, wherein the conductive material is selected from metals, conductive metal oxides, and conductive forms of carbon, and wherein the conductive polymeric material is an intrinsically conducting polymer (ICP).

18. The electrode assembly of claim 16, wherein the shear modulus of the conductive material is at least 2 times the shear modulus of the conductive polymeric material.

19. The electrode assembly of claim 16 as incorporated into a biosensor for detecting the concentration of an analyte in a patient.

20. A biosensor for detecting an analyte, the biosensor comprising a multilayer electrochemical sensor, the multilayer electrochemical sensor comprising a substrate, a layer of conductive polymeric material disposed on the substrate, and a layer of conductive material disposed on the layer of conductive polymeric material.

21. The biosensor of claim 20, comprising a control unit in electrical communication with the electrochemical sensors.

22. A method for manufacturing an electrode assembly, the method comprising forming an electrode pattern in a multilayer structure, the multilayer structure comprising an intrinsically conductive polymer (ICP) disposed on a substrate and a conductive material disposed on the ICP, wherein the conductive material is selected from metals, conductive metal oxides, and conductive forms of carbon.

23. The method of claim 22, comprising depositing the ICP on the substrate and depositing the conductive material on the ICP prior to forming the electrode pattern.

24. The method of claim 22, wherein the electrode pattern comprises a working electrode, a counter electrode, a trace associated with the working electrode, and a trace associated with the counter electrode.

25. The method of claim 24, wherein the electrode pattern comprises an electrical contact associated with the working electrode and an electrical contact associated with the counter electrode, wherein the electrical contacts are configured to contact a control unit.

26. The method of claim 22, wherein the electrode assembly is suitable for measuring an analyte concentration in a liquid.
